(19) United States
(12) Patent Application Publication
(10) Pub. No.: US 2026/0207153 A1
(43) Pub. Date: Jul. 23, 2026

(54) RADIATION IMAGING APPARATUS

(71) Applicant: CANON KABUSHIKI KAISHA, Tokyo (JP)

(72) Inventor: YURI YOSHIMURA, Tokyo (JP)

(21) Appl. No.: 19/437,983

(22) Filed: Dec. 31, 2025

(30) Foreign Application Priority Data
Jan. 17, 2025 (JP) 2025-006942

(51) Int. Cl. A61B 6/50; A61B 6/42

(52) U.S. Cl. CPC A61B 6/502; A61B 6/4283

(57) ABSTRACT

A radiation imaging apparatus includes a radiation detector and a housing that accommodates the detector. The housing has an entrance surface on which radiation is incident, a rear surface opposite the entrance surface, and a plurality of side surfaces connecting them. At least one side surface includes a fastening portion at which a plurality of members constituting the housing are connected and, on the at least one side surface, (a) a first inner-wall portion located on an entrance-surface side and closer to an outermost surface of the at least one side surface, and (b) a second inner-wall portion located on a rear-surface side of the first inner-wall portion and including the fastening portion, are provided. An end portion of the radiation detector on the at least one side-surface side is disposed opposite the first inner-wall portion and is closer to the outermost surface than the second inner-wall portion.

### BACKGROUND

### Field of the Technology

The present disclosure relates to a radiation imaging apparatus.

### Description of the Related Art

Radiation imaging apparatuses that detect the intensity distribution of radiation transmitted through a subject to provide a radiographic image are used in medical diagnosis and industrial non-destructive inspection. In a mammography device equipped with such a radiation imaging apparatus, radiation imaging is performed while a breast of the subject is compressed and flattened between plates.

As illustrated in FIG. 1, in a radiation imaging apparatus housed in a mammography device, a distance from the device side surface configured to contact the chest wall of the subject to the effective imaging region is to be minimized. Otherwise, a radiographic image may not be obtained within this distance, potentially leading to not detecting a lesion or the like in an area near the chest wall of the subject. Generally, this distance is referred to as the chest-wall edge non-imaging distance.

Japanese Patent No. 5908668 discusses a radiation imaging apparatus provided with a housing structure composed of a radiation entrance surface member, a rear surface member facing the radiation entrance surface member, and a side surface member. However, this side surface member is a thick member covering the periphery of the effective imaging region, so that the chest-wall edge non-imaging distance is large. If this side surface member is made thinner, the rigidity of the entire apparatus will be reduced.

### SUMMARY

The present disclosure provides a radiation imaging apparatus that provides radiographic images effective in a wide range with a short chest-wall edge non-imaging distance, without reducing the rigidity of the entire apparatus.

An aspect of the present disclosure provides a radiation imaging apparatus that includes a radiation detector, and a housing configured to accommodate the radiation detector, the housing having an entrance surface on which radiation is incident, a rear surface opposite to the entrance surface, and a plurality of side surfaces connecting the entrance surface and the rear surface. At least one of the plurality of side surfaces includes a fastening portion at which a plurality of members constituting the housing are connected and, on at least one side surface of the plurality of side surfaces, a first inner-wall portion and a second inner-wall portion are provided. The first inner-wall portion is located on an entrance-surface side, closer to an outermost surface of the at least one side surface. The second inner-wall portion is located on a rear-surface side of the first inner-wall portion, including the fastening portion. An end portion of the radiation detector on a side of the at least one side surface is opposite to the first inner-wall portion, closer to the outermost surface of the at least one side surface than the second inner-wall portion.

Features of the present disclosure will become apparent from the following description of embodiments with reference to the attached drawings. The following description of embodiments is described by way of example.

### BRIEF DESCRIPTION OF THE DRAWINGS

FIG. 1 is a schematic diagram illustrating a general configuration of a mammography device.

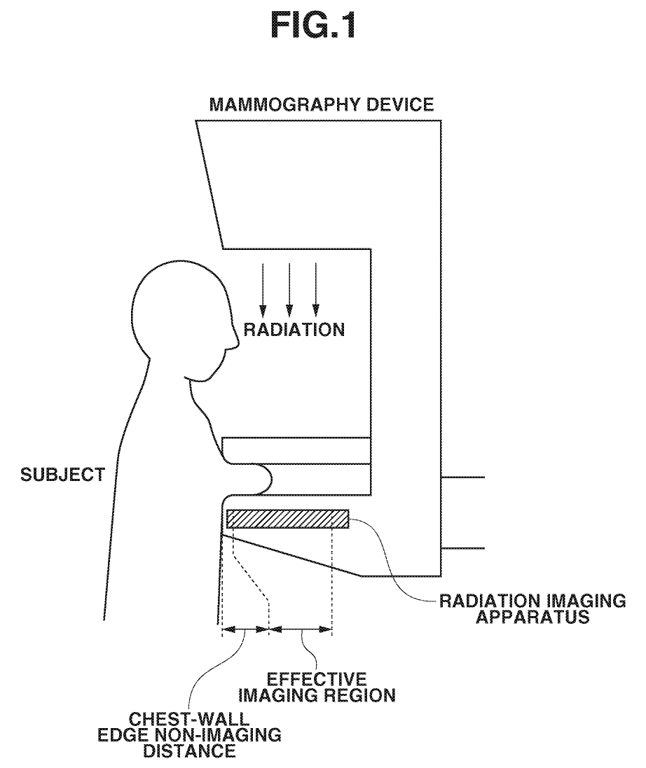

FIG. 2 is a top perspective view illustrating an external appearance of a radiation imaging apparatus according to an embodiment.

FIG. 3 is a top perspective view illustrating the radiation imaging apparatus according to the present embodiment in an exploded state.

FIG. 4 is a partially omitted cross-sectional view taken along a dashed line B-B in FIG. 2.

FIG. 5 is a cross-sectional view illustrating a narrow-frame side surface of a housing in the radiation imaging apparatus according to the embodiment.

FIG. 6 is a cross-sectional view illustrating a narrow-frame side surface of a housing in a radiation imaging apparatus according to a first modification.

FIG. 7 is a cross-sectional view illustrating a narrow-frame side surface of a housing in a radiation imaging apparatus according to a second modification.

FIG. 8 is a cross-sectional view illustrating a narrow-frame side surface of a housing in a radiation imaging apparatus according to a third modification.

FIG. 9 is a cross-sectional view illustrating a narrow-frame side surface of a housing in a radiation imaging apparatus according to a fourth modification.

### DESCRIPTION OF THE EMBODIMENTS

Hereinafter, an embodiment will be described in detail with reference to the drawings. In the following description, components common to multiple drawings are denoted by common reference numerals. Common components in a plurality of drawings may thus be described by cross-reference, and the description of components with common reference numerals may be incorporated by reference as appropriate. The details of dimensions and structures described in the present embodiment are not limited to those described in the text and drawings. Herein, radiation may include α-rays, β-rays, γ-rays, particle beams, and cosmic rays, as well as X-rays.

### Configuration of Radiation Imaging Apparatus 100

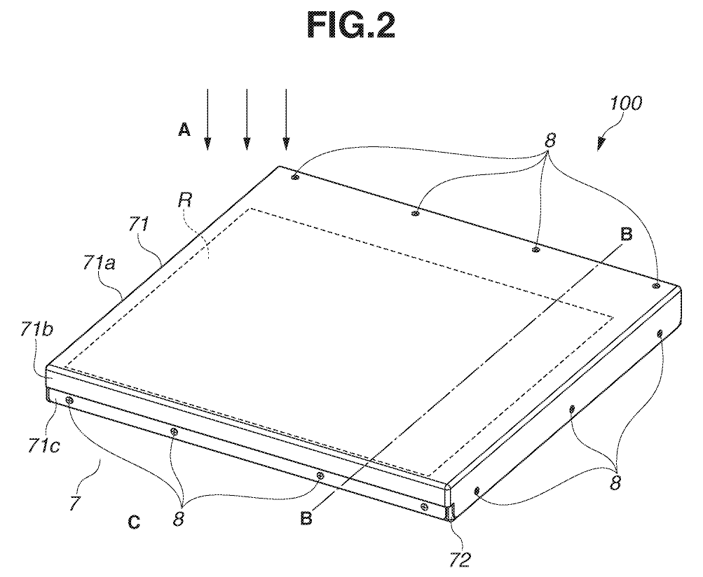

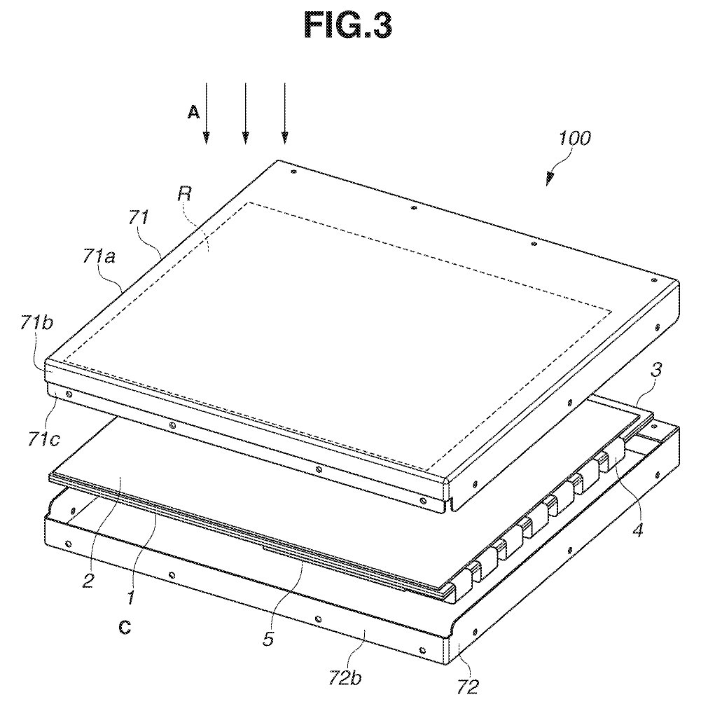

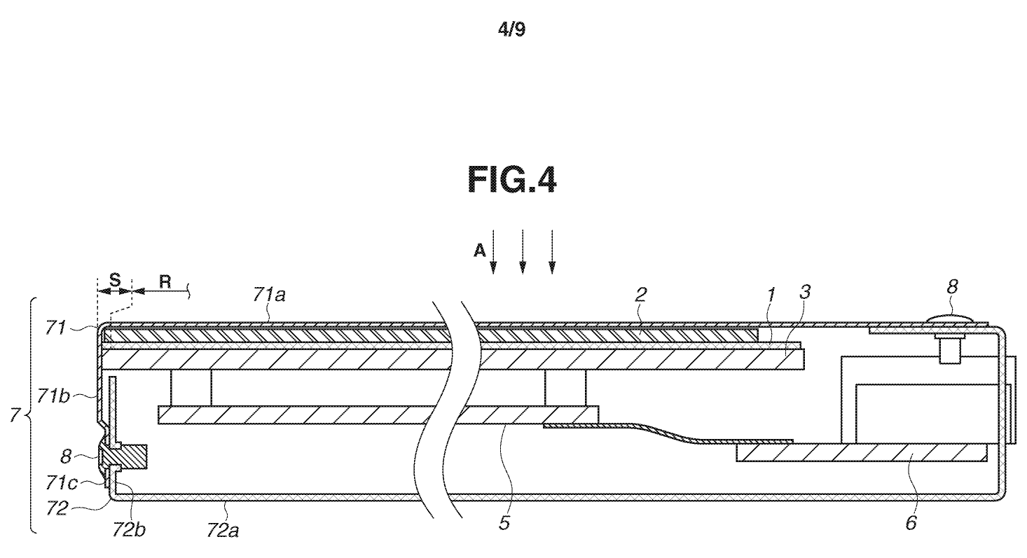

Hereinafter, a schematic configuration of a radiation imaging apparatus 100 according to the present embodiment will be described with reference to FIGS. 2 to 4. The radiation imaging apparatus 100 is used, for example, for mammography imaging. FIG. 2 is a top perspective view illustrating an external appearance of the radiation imaging apparatus 100 according to the present embodiment. FIG. 3 is a top perspective view illustrating the radiation imaging apparatus 100 according to the present embodiment in an exploded state. FIG. 4 is a partially omitted cross-sectional view taken along a dashed line B-B of FIG. 2.

The radiation imaging apparatus 100 is irradiated by a radiation generator, and then generates a radiographic image corresponding to the radiation transmitted through the subject. The radiation imaging apparatus 100 transfers the generated radiographic image to an external device, and the transferred radiographic image is displayed on an external display device or the like.

The radiation imaging apparatus 100 includes a radiation detection panel 1 as a radiation detector for converting radiation into an electrical signal. The radiation detection panel 1 has a function of converting radiation incident from an A-side surface into an electrical signal. Hereinafter, the A-side surface is referred to as the entrance surface, and the surface opposite to the entrance surface is referred to as the rear surface. The A-side herein is the upper side of the radiation imaging apparatus 100. The radiation detection panel 1 includes a sensor substrate in which a plurality of photoelectric conversion elements (sensors) is arranged in a two-dimensional manner on a glass substrate, a phosphor layer disposed on the sensor substrate, and a phosphor protective film disposed on the phosphor layer. The phosphor protective film is made of a material with relatively high moisture resistance and is used to protect the phosphor layer.

In the radiation detection panel 1 with the above configuration, the phosphor layer emits light in response to the incident radiation, and the emitted light is converted into an electrical signal by the photoelectric conversion elements arranged on the sensor substrate. In the radiation detection panel 1, a part or the entirety of the region of the photoelectric conversion elements is defined as an effective imaging region R. The effective imaging region R is a region where radiation imaging can be performed and an image is actually generated. However, the configuration of the radiation detection panel 1 is not limited to that described here; for example, a plurality of phosphor layers may be provided, or a direct conversion element that directly converts radiation into an electrical signal may be employed instead of the phosphor layer and the photoelectric conversion elements. The material of the sensor substrate of the radiation detection panel 1 is not limited to glass, and may be a more flexible resin material or another kind of material.

The radiation detection panel 1 is electrically connected to a control board 5 via a plurality of flexible circuit boards 4. The control board 5 reads the electrical signals converted in the radiation detection panel 1 to perform various processing. For example, the control board 5 converts the electrical signals into digital signals to generate radiographic image data. The control board 5 transfers the generated radiographic image data to an external device via an external connection unit 6. The external connection unit 6 includes a board for exchanging various kinds of control data, transmitting radiographic image data, and receiving power, and various types of connectors.

A cushioning material 2 is provided between the housing 7, which will be described below, and the radiation detection panel 1, to protect the radiation detection panel 1 from external forces. If the radiation detection panel 1 is less likely to be damaged under expected external force conditions, the cushioning material 2 may be omitted. A support base 3 supports the radiation detection panel 1 on the rear surface side of the housing 7 and supports both the control board 5 and the external connection unit 6 or part of them on the rear surface side.

The housing 7 encloses the above-described structural components. In each of the figures, the letter C indicates the side surface of the housing 7 facing the chest wall of the subject in the radiation imaging apparatus 100. That is, the letter C indicates the side facing the chest wall of the subject in the radiation imaging apparatus 100 housed in the mammography device. In the present embodiment, out of the distances from the outermost ends of the side surfaces of the periphery of the housing 7 to the effective imaging region R, the distance on the C side is the smallest, which corresponds to the narrowest frame. That is, this distance is a chest-wall edge non-imaging distance S in the radiation imaging apparatus 100. Hereinafter, the C-side surface of the housing 7 is referred to as the narrow-frame side surface.

The housing 7 includes a first housing member 71 and a second housing member 72.

The first housing member 71 mainly covers the effective imaging region R on the entrance surface of the radiation imaging apparatus 100, and also covers the entirety or part of the side surfaces including the narrow-frame side surface in addition to the entrance surface.

The entrance surface of the first housing member 71 may include a first housing side wall integrally formed with a first housing entrance surface 71a. As the material for the first housing member 71, a material that has radiolucency, such as Carbon Fiber Reinforced Plastics (CFRP), and that can provide relatively high rigidity even with small thickness.

A typical molding method for CFRP is laminating sheet-like base materials and then pressing or heat-bonding the base materials. This method provides a member with substantially uniform thickness determined by the number of laminated sheets. Other methods include injection molding of resin kneaded with discontinuous carbon fibers.

As the material for the first housing member 71, various kinds of resins other than CFRP, such as polycarbonate or acrylic, may be used.

The second housing member 72 mainly covers the rear surface of the radiation imaging apparatus 100, and also covers the entirety or part of the side surfaces including the narrow-frame side surface and the entrance surface in addition to the rear surface. The rear surface portion of the second housing member 72 may be referred to as a second housing rear surface 72a. The second housing member 72 may have higher rigidity than the first housing member 71. As the material for the second housing member 72, a metal having high rigidity, such as iron, aluminum, or magnesium may be used. The second housing member 72 is responsible for the overall rigidity of the radiation imaging apparatus 100 and also serves to protect the interior from external noise and to block radiation that has passed through the radiation detection panel 1 during imaging. If external noise protection or radiation shielding is performed by other means, the material of the second housing member 72 may be a high-rigidity resin or the like instead of metal.

The first housing member 71 and the second housing member 72 are fastened together with connecting members, such as a plurality of screws 8, at portions near the rear surface within the narrow-frame side surface, at portions distant from the narrow-frame side surface within the entrance surface, and at portions near the rear surface within the two side surfaces adjacent to the narrow-frame side surface.

An insulating layer 9, as an insulating sheet material, is provided on an inner side of the first housing member 71. As described below, on the narrow-frame side surface, the first housing member 71 and the support base 3 are positioned close to each other via the insulating layer 9, and the radiation detection panel 1, the support base 3, and the first housing member 71 are electrically insulated from each other. The insulating layer 9 may adhere to the inner surface of the first housing member 71, and be integrally formed with the first housing member 71, or a coating film may be provided on the inner surface thereof instead of a sheet material to exhibit electrical insulation property. The insulating layer 9 may also be formed on an end surface close to the narrow frame of the radiation detection panel 1 or the support base 3, or both, rather than on the first housing member 71. The electrical insulation between the first housing member 71 and the support base 3 provides improved image quality of the captured image. If the desired image quality is provided, the insulating layer 9 may be omitted.

### Configuration of the Narrow-Frame Side Surface

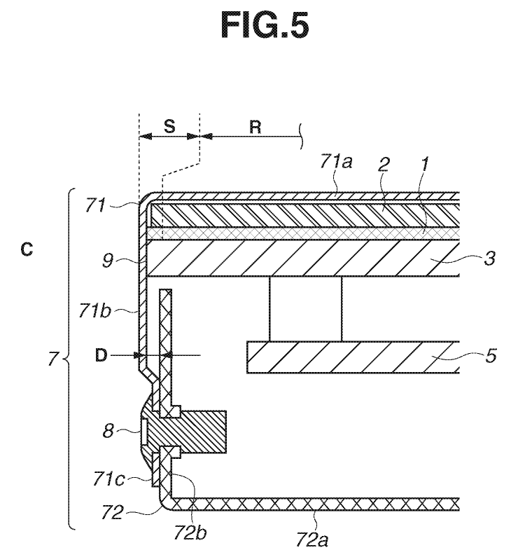

Hereinafter, the configuration of the narrow-frame side surface of the housing 7, which is a feature of the radiation imaging apparatus 100, will be described in detail. FIG. 5 is a cross-sectional view illustrating the narrow-frame side surface of the housing 7 in the radiation imaging apparatus 100.

On the narrow-frame side surface of the housing 7, the first housing member 71 has a first housing side wall integrally formed with the first housing entrance surface 71a. The first housing side wall includes a first-housing upper wall 71b near the entrance surface of the housing 7 and a first-housing lower wall 71c near the rear surface of the housing 7. The first-housing upper wall 71b and the first-housing lower wall 71c extend in a radiation incident direction and are substantially parallel to each other. The step portion between the first-housing upper wall 71b and the first-housing lower wall 71c is connected by a gently sloping surface. The first-housing upper wall 71b may be thinner than either the first housing entrance surface 71a or the first-housing lower wall 71c, or both. With improved dimensional stability and reduced manufacturing cost taken into consideration, a uniformly thick high-rigidity material, such as CFRP, may be used to form the entire first housing member 71.

On the other hand, on the narrow-frame side surface of the housing 7, the second housing member 72 has a second housing side wall 72b integrally formed with the second housing rear surface 72a. On the narrow-frame side surface, the outer first housing member 71 and the inner second housing side wall 72b are connected. This fastening portion (connection portion) is spaced apart from the radiation detection panel 1 and the support base 3 supporting the radiation detection panel 1 and positioned near the rear surface of the housing 7, and the end portion of the radiation detection panel 1 on the narrow-frame side extends from the second housing side wall 72b toward the first-housing upper wall 71b. In the present embodiment, the first housing member 71 and the second housing side wall 72b are partially overlapped on the narrow-frame side surface and are fastened together at the overlapped portion, and this overlapped portion is spaced apart from the radiation detection panel 1 and the support base 3 and positioned near the rear surface of the housing 7. Such a configuration provides a narrow frame property of the narrow-frame side surface as the chest-wall surface of the housing 7, reducing the chest-wall edge non-imaging distance S, which is the distance from the first-housing upper wall 71b to the effective imaging region R of the radiation detection panel 1.

On the narrow-frame side surface, the inner surface of the first-housing upper wall 71b is located closer to the exterior of the housing 7 than the inner surface of the first-housing lower wall 71c, and a step is formed between the inner surfaces of the first-housing upper wall 71b and the first-housing lower wall 71c. As a result, the outer surface of the first-housing upper wall 71b protrudes further outward from the housing 7 than the outer surface of the first-housing lower wall 71c and constitutes an outermost surface on the narrow-frame side surface of the housing 7. The fastening portion is provided on the first housing-lower wall 71c, and the first-housing lower wall 71c and the second housing side wall 72b are fastened and fixed together with a screw 8.

As described above, the fastening portion with the screw 8 between the first-housing lower wall 71c and the second housing side wall 72b is spaced apart from the support base 3 and positioned near the rear surface of the housing 7. Here, for example, if the fastening portion is provided on the first housing entrance surface 71a or an upper part of the narrow-frame side surface, the radiation detection panel 1 and the support base 3 are spaced apart from the narrow-frame side surface to secure space for screw fastening, resulting in an increased chest-wall edge non-imaging distance S. In the present embodiment, the entrance surface portion and the narrow-frame side surface portion of the first housing member 71 are formed as a continuous single member, and the fastening portion with the second housing member 72 is disposed on the rear surface side away from the support base 3. This provides the support base 3 at a position in contact with the insulating layer 9 on the inner surface of the first-housing upper wall 71b, the position of which is close to the narrow-frame side surface, providing a narrower frame.

The head of the screw 8 (the end on the narrow-frame side surface) is either flush with the outermost surface of the narrow-frame side surface (in the present embodiment, the outer surface of the first-housing upper wall 71b) or recessed toward the inside of the housing 7 from the outermost surface of the narrow-frame side surface. That is, the head of the screw 8 does not protrude from the first-housing upper wall 71b. If the screw head protrudes, the chest-wall edge non-imaging distance S will increase by the amount of the protrusion. In the present embodiment, even though the screw 8 is provided on the narrow-frame side surface, the outer surface of the first-housing upper wall 71b serves as the chest-wall surface configured to contact the chest wall of the subject, and the chest-wall edge non-imaging distance S can be reduced.

There are no particular restrictions on the screws 8 used in the present embodiment, and, for example, pan head, binding head, or low head screws can be used.

For example, using countersunk screws as the screws 8 prevents the screw heads from protruding from the first-housing upper wall 71b on the narrow-frame side surface without providing a step. In this case, the first-housing lower wall 71c needs to have a thickness sufficient for countersinking. Specifically, a thickness of about 1 mm or more is provided.

In the present embodiment, a step amount D between the inner surfaces of the first-housing upper wall 71b and the first-housing lower wall 71c is ensured, and screws that do not require countersinking are used as the screws 8. This allows the thickness of the first housing member 71 to be reduced to less than 1 mm, for example, about 0.4 mm to about 0.8 mm, providing a narrower frame. However, making the thickness of the first housing member 71 different depends on the surface and use countersunk screws.

In addition, to prevent the head of the screw 8 from protruding from the first-housing upper wall 71b with the uniformly thick first housing member 71, the first-housing lower wall 71c and the second housing side wall 72b may be overlapped with the first-housing lower wall 71c in an inclined shape with respect to the first-housing upper wall 71b. However, in general, regardless of the molding method or material, an inclined surface with respect to the main surface of a member has low dimensional accuracy in terms of surface angle or the like, which makes it difficult to closely fit inclined surfaces of different members, making it unsuitable for screw fastening. Thus, a step may be provided on the narrow-frame side surface as in the present embodiment.

In the present embodiment described above, the entire first housing member 71 is formed of a high-rigidity material, such as CFRP, with a thin uniform thickness. The overall rigidity of the housing 7 is thus provided by the second housing member 72.

Thus, the second housing member 72 may be made of a material and shape that exhibits higher rigidity than the first housing member 71. High material rigidity specifically means that material property values, such as Young’s modulus, tensile strength, flexural strength, and compressive strength, are high. Even if the material property values are equivalent or somewhat inferior, the rigidity can be increased by increasing the basic thickness of the second housing member 72 or providing reinforcing shapes, such as ribs.

As a method of connecting the first housing member 71 and the second housing member 72, in consideration of integration of rigidity and ensuring ability of disassembly for maintenance and the like, screw fastening as in the present embodiment is desirable rather than adhesion or the like. Although slightly inferior to screws in terms of ability of disassembly and fastening strength, rivets or the like may also be used. In this case, the height of the screw head described above is replaced with the height of the rivet head, and the step amount D is set accordingly.

On the narrow-frame side surface of the housing 7, since the support base 3 is present up to a position in contact with the insulating layer 9, the upper end of the second housing side wall 72b is positioned spaced from the support base 3 and near the rear surface. This allows the radiation detection panel 1 to be provided closer to the narrow-frame side surface than the second housing side wall 72b. On the other hand, as illustrated in FIG. 3, on the two side surfaces adjacent to the narrow-frame side surface, the upper end of the second housing member 72 is present up to near the entrance surface, and the side surface as a surface of the second housing member 72 opposite to the narrow-frame side surface also has a surface on part of the entrance surface. Thus, even if the upper end of the second housing side wall 72b on the narrow-frame side surface is at a position lower than the support base 3, the radiation imaging apparatus 100 has sufficient rigidity against the load from the entrance surface side.

As described above, according to the present embodiment, the radiation imaging apparatus 100 provides effective radiographic images over a wide range by minimizing the chest-wall edge non-imaging distance S without reducing the rigidity of the entire apparatus.

### Various Modifications of the Present Embodiment

Hereinafter, various modifications of the present embodiment will be described. In the modifications, a radiation imaging apparatus 100 is disclosed in a similar manner to the disclosure in the present embodiment, but differs from the present embodiment in the form of connection between the first-housing lower wall 71c and the second housing member 72 on the narrow-frame side surface of the housing 7. For conciseness, descriptions of components and the like similar to those in the radiation imaging apparatus 100 of the present embodiment are incorporated by reference herein.

### First Modification

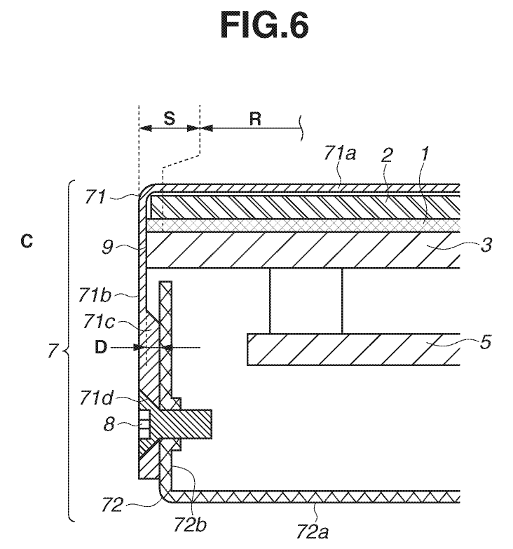

FIG. 6 is a cross-sectional view illustrating a narrow-frame side surface of the housing 7 in a radiation imaging apparatus 100 according to a first modification.

In the first modification, outer surfaces of both a first-housing upper wall 71b and a first housing-lower wall 71c in the first housing member 71 are integrally formed to constitute an outermost surface of the narrow-frame side surface.

The first-housing upper wall 71b is formed thinly as in the present embodiment. On the other hand, the first-housing lower wall 71c is formed to be thicker than the first-housing upper wall 71b and is overlapped and connected to a second housing side wall 72b of the second housing member 72. The difference in thickness between the first-housing lower wall 71c and the first-housing upper wall 71b is a step amount D between inner surfaces of the first-housing upper wall 71b and the first-housing lower wall 71c. The first-housing lower wall 71c has a thickness sufficient to countersink, so that a countersunk hole 71d that is a through hole is formed at the fastening portion connecting to the second housing side wall 72b, and the first-housing lower wall 71c and the second housing side wall 72b are fastened together with a screw 8 that is a countersunk screw.

Here, the first housing entrance surface 71a may be the same in thickness as either the first-housing upper wall 71b or the first-housing lower wall 71c, or both, or may be different from both, and can be freely set based on the strength required for the first housing entrance surface 71a. However, if the thickness of the first housing member 71 is made non-uniform, it is necessary to devise process methods, such as changing the number of sheets laminated of the CFRP base material depending on the surface, using injection-molded materials, or integrally forming separate members.

The fastening portion between the first-housing lower wall 71c and the second housing side wall 72b is spaced apart from the radiation detection panel 1 and the support base 3 supporting the radiation detection panel 1 and positioned near the rear surface of the housing 7. The end portion of the radiation detection panel 1 on the narrow-frame side extends from the second housing side wall 72b toward the first-housing upper wall 71b. In the first modification, the first housing member 71 and the second housing side wall 72b are partially overlapped on the narrow-frame side surface and are fastened together with the screw 8 at the overlapped portion, and this overlapped portion is spaced apart from the radiation detection panel 1 and the support base 3 and positioned near the rear surface of the housing 7. Such a configuration provides a narrow frame property of the narrow-frame side surface as the chest-wall surface of the housing 7, and allows reduction of the chest-wall edge non-imaging distance S, which is the distance from the first-housing upper wall 71b to the effective imaging region R of the radiation detection panel 1.

In addition, in the first modification, the thickness of the first-housing lower wall 71c ensures the step amount D sufficient for the head of a countersunk screw not to protrude from the surface of the first-housing lower wall 71c, and the countersunk hole 71d can be formed deep enough to use a countersunk screw as the screw 8. This allows the first-housing lower wall 71c and the second housing side wall 72b to be fastened together with a countersunk screw so that the head of the countersunk screw does not protrude from the surface of the first-housing lower wall 71c. Such a configuration provides the narrow-frame side surface of the housing 7 serving as the chest-wall surface configured to contact the chest wall of a subject while the head of the screw 8 does not interfere, and allows reduction of the chest-wall edge non-imaging distance S.

### Second Modification

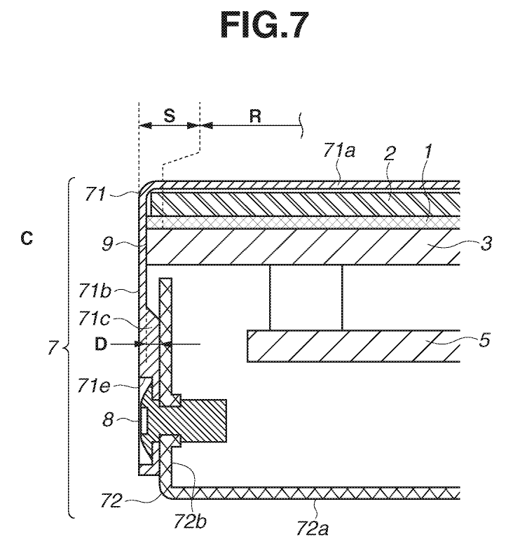

FIG. 7 is a cross-sectional view illustrating a narrow-frame side surface of the housing 7 in a radiation imaging apparatus 100 according to a second modification.

In the second modification, a first housing member 71 and a second housing member 72 are formed in a similar manner to those in the first modification described above. That is, a first-housing upper wall 71b is formed thinly as in the present embodiment, a first-housing lower wall 71c is formed to be thicker than the first-housing upper wall 71b, and is overlapped and connected to a second housing side wall 72b of the second housing member 72. The difference in thickness between the first-housing lower wall 71c and the first-housing upper wall 71b is a step amount D between inner surfaces of the first-housing upper wall 71b and the first-housing lower wall 71c.

The first-housing lower wall 71c has a thickness sufficient to counterbore a counterbored hole 71e, which is a blind hole formed at the fastening portion connecting to the second housing side wall 72b, and the first-housing lower wall 71c and the second housing side wall 72b are fastened together with a screw 8 as in the present embodiment.

The fastening portion between the first-housing lower wall 71c and the second housing side wall 72b is spaced apart from the radiation detection panel 1 and the support base 3 supporting the radiation detection panel 1 and positioned near the rear surface of the housing 7. The end portion of the radiation detection panel 1 on the narrow-frame side extends from the second housing side wall 72b toward the first-housing upper wall 71b. In the second modification, the first housing member 71 and the second housing side wall 72b are partially overlapped on the narrow-frame side surface and are fastened together with the screw 8 at the overlapped portion, and this overlapped portion is spaced apart from the radiation detection panel 1 and the support base 3 and positioned near the rear surface of the housing 7. Such a configuration provides a narrow frame property of the narrow-frame side surface as the chest-wall surface of the housing 7, and allows reduction of the chest-wall edge non-imaging distance S, which is the distance from the first-housing upper wall 71b to the effective imaging region R of the radiation detection panel 1.

In addition, in the second modification, the thickness of the first-housing lower wall 71c ensures the step amount D sufficient for the head of the screw 8 not to protrude from the surface of the first-housing lower wall 71c, and the counterbored hole 71e can be formed deep enough to use the screw 8. This allows the first-housing lower wall 71c and the second housing side wall 72b to be fastened together with the screw 8 while the head of the screw 8 does not protrude from the surface of the first-housing lower wall 71c. Such a configuration provides the narrow-frame side surface of the housing 7 serving as the chest-wall surface configured to contact the chest wall of the subject while the head of the screw 8 does not interfere, and allows reduction of the chest-wall edge non-imaging distance S.

### Third Modification

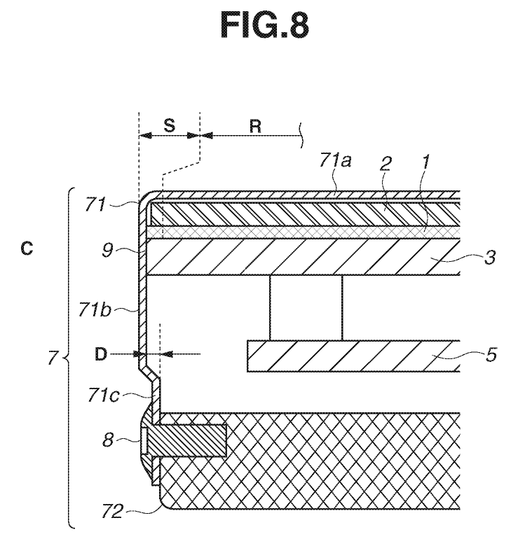

FIG. 8 is a cross-sectional view illustrating a narrow-frame side surface of a housing 7 in a radiation imaging apparatus 100 according to a third modification.

In the third modification, the components other than a second housing member 72 are formed in the same manner as in the present embodiment.

In a housing 7, the entire first housing member 71 is formed of a high-rigidity material, such as CFRP, with a small uniform thickness, so that the overall rigidity of the housing 7 is provided by the second housing member 72. In the third modification, the second housing member 72 thus is formed of a high-rigidity material with a greater thickness than that of the first housing member 71. As in the present embodiment, a metal, such as iron, aluminum, or magnesium, may be used as the high-rigidity material. The second housing member 72 is a uniformly-thick integral member with a thickness sufficient to be fastened with the first-housing lower wall 71c and the screw 8 at the end face on the narrow-frame side surface.

According to the third modification, the radiation imaging apparatus 100 provides effective radiographic images over a wide range by minimizing the chest-wall edge non-imaging distance S while maintaining sufficient rigidity of the entire apparatus.

### Fourth Modification

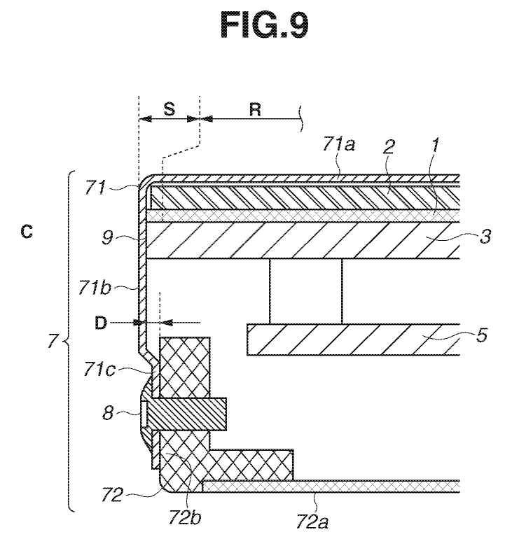

FIG. 9 is a cross-sectional view illustrating a narrow-frame side surface of a housing 7 in a radiation imaging apparatus 100 according to a fourth modification.

In the fourth modification, the components other than a second housing member 72 are formed in the same manner as in the present embodiment.

In the housing 7, the entire first housing member 71 is formed of a high-rigidity material, such as CFRP, with a thin uniform thickness, so that the overall rigidity of the housing 7 is provided by the second housing member 72. In the fourth modification, a second housing side wall 72b of the second housing member 72 is formed of a high-rigidity material with a greater thickness than that of the first housing member 71. As in the present embodiment, a metal, such as iron, aluminum, or magnesium, may be used as the high-rigidity material. The second housing rear surface 72a of the second housing member 72 is the same as that of the present embodiment. In this way, the second housing member 72 is composed of two members: the thin second housing rear surface 72a and the thick second housing side wall 72b, and the first-housing lower wall 71c and the second housing side wall 72b are fastened together with the screw 8 at the end face on the narrow-frame side surface.

According to the fourth modification, the radiation imaging apparatus 100 provides effective radiographic images over a wide range by minimizing the chest-wall edge non-imaging distance S while maintaining sufficient rigidity of the entire apparatus.

The present disclosure includes the following configurations.

### Configuration 1

A radiation imaging apparatus is provided that includes a radiation detector configured to convert radiation transmitted through a subject into an electrical signal and a housing configured to accommodate the radiation detector. The housing includes an entrance surface on which the radiation is incident, a rear surface opposite to the entrance surface, and a plurality of side surfaces connecting the entrance surface and the rear surface. At least one side surface of the housing includes a fastening portion at which a first housing side wall of the first housing member and a second housing side wall of the second housing member are connected in a radiation incident direction. The fastening portion is disposed inside the housing, spaced from the radiation detector and near the rear surface. An end portion of the radiation detector on the at least one side of the side-surface extends from a second housing side wall toward a first housing side wall.

### Configuration 2

The radiation imaging apparatus according to Configuration 1, wherein the first housing side wall and the second housing side wall are partially overlapped with each other, and an overlapped fastening portion is positioned spaced apart from the radiation detector and near the rear surface inside the housing.

### Configuration 3

The radiation imaging apparatus according to Configuration 1 or 2, wherein the first housing side wall includes a first-housing upper wall extending in the radiation incident direction and a first-housing lower wall positioned closer to the rear surface than the first-housing upper wall, wherein an inner surface of the first-housing upper wall is positioned further outward from the housing than the inner surface of the first-housing lower wall, wherein an outer surface of the first-housing upper wall constitutes an outermost surface of the at least one side surface, and wherein the first-housing lower wall is joined to the second housing side wall at the fastening portion.

### Configuration 4

The radiation imaging apparatus according to Configuration 3, wherein the outer surface of the first-housing upper wall protrudes outward from the housing beyond an outer surface of the first-housing lower wall.

### Configuration 5

The radiation imaging apparatus according to Configuration 3, wherein the first-housing lower wall is thicker than the first-housing upper wall, and the outer surface of the first-housing lower wall and the outer surface of the first-housing upper wall constitute the outermost surface of the at least one side surface.

### Configuration 6

The radiation imaging apparatus according to Configuration 5, wherein a through-hole is formed in the first-housing lower wall at the fastening portion.

### Configuration 7

The radiation imaging apparatus according to Configuration 5, wherein a blind hole is formed in the first-housing lower wall at the fastening portion.

### Configuration 8

The radiation imaging apparatus according to any one of Configurations 2 to 7, wherein the fastening portion is fastened with a connection member, and an end of the connection member on the side of the at least one side-surface is flush with, or recessed toward an inside of the housing from, the outermost surface of the at least one side surface.

### Configuration 9

The radiation imaging apparatus according to any one of Configurations 3 to 8, further comprising a support base configured to support the radiation detector on a surface on the rear-surface side, wherein an end face of the support base on the side of the at least one side-surface is positioned on an inner surface of the first-housing upper wall, and wherein an upper end of the second housing side wall on an entrance-surface side is spaced from the support base and near the rear surface.

### Configuration 10

The radiation imaging apparatus according to Configuration 9, wherein an insulating layer is provided on the inner surface of the first-housing upper wall, and the end face of the support base on the side of the at least one side-surface is in contact with the insulating layer.

### Configuration 11

The radiation imaging apparatus according to any one of Configurations 3 to 10, wherein the first-housing upper wall is thinner than either the entrance surface of the first housing member or the first-housing lower wall, or both.

### Configuration 12

The radiation imaging apparatus according to any one of Configurations 3 to 10, wherein the first housing member has a uniform thickness.

### Configuration 13

The radiation imaging apparatus according to any one of Configurations 3 to 12, wherein the first housing member has a thickness less than 1 mm.

### Configuration 14

The radiation imaging apparatus according to any one of Configurations 1 to 13, wherein the second housing member has higher rigidity than the first housing member.

### Configuration 15

The radiation imaging apparatus according to Configuration 14, wherein the second housing member is an integral member with uniform thickness that is thicker than the first housing member.

### Configuration 16

The radiation imaging apparatus according to Configuration 14, wherein the second housing member includes a second-housing rear surface forming the rear surface of the housing and a second-housing side wall thicker than the first housing member.

### Configuration 17

The radiation imaging apparatus according to any one of Configurations 1 to 16, wherein the at least one side surface of the housing is a narrow-frame side surface in which a distance from an outer surface of the housing to an effective imaging region of the radiation detector is smaller than that of another side surface of the housing.

### Configuration 18

The radiation imaging apparatus according to any one of Configurations 1 to 17, wherein the radiation imaging apparatus is used for mammography imaging.

### Other Embodiments

Embodiment(s) of the present disclosure can also be realized by a computer of a system or apparatus that reads out and executes computer executable instructions (e.g., one or more programs) recorded on a storage medium (which may also be referred to as a non-transitory computer-readable storage medium) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)^{TM}), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-006942, filed January 17, 2025, which is hereby incorporated by reference herein in its entirety.

What is claimed is:

1. A radiation imaging apparatus comprising:
a radiation detector; and
a housing configured to accommodate the radiation detector, the housing having an entrance surface on which radiation is incident, a rear surface opposite to the entrance surface, and a plurality of side surfaces connecting the entrance surface and the rear surface,
wherein at least one of the plurality of side surfaces includes a fastening portion at which a plurality of members constituting the housing are connected and, on at least one side surface of the plurality of side surfaces, a first inner-wall portion and a second inner-wall portion are provided,
wherein the first inner-wall portion is located on an entrance-surface side, closer to an outermost surface of the at least one side surface,
wherein the second inner-wall portion is located on a rear-surface side of the first inner-wall portion, including the fastening portion, and
wherein an end portion of the radiation detector on a side of the at least one side surface is opposite to the first inner-wall portion, closer to the outermost surface of the at least one side surface than the second inner-wall portion.

2. The radiation imaging apparatus according to claim 1, wherein the plurality of members includes a first housing member and a second housing member, and
wherein side walls of the first housing member and the second housing member are partially overlapped with each other, and
wherein an overlapped fastening portion is spaced apart from the radiation detector, toward the rear-surface side.

3. The radiation imaging apparatus according to claim 2, wherein a side wall of the first housing member includes a first-housing upper wall extending in a radiation incident direction and a first-housing lower wall located closer to the rear surface than the first-housing upper wall,
wherein an outer surface of the first-housing upper wall constitutes the outermost surface of the at least one side surface, and
wherein the first-housing lower wall is joined to a side wall of the second housing member at the fastening portion.

4. The radiation imaging apparatus according to claim 3, wherein an outer surface of the first-housing upper wall protrudes outward from the housing beyond an outer surface of the first-housing lower wall.

5. The radiation imaging apparatus according to claim 3, wherein the first-housing lower wall is thicker than the first-housing upper wall, and the outer surface of the first-housing lower wall and the outer surface the first-housing upper wall constitute the outermost surface of the at least one side surface.

6. The radiation imaging apparatus according to claim 5, further comprising a through-hole formed in the first-housing lower wall at the fastening portion.

7. The radiation imaging apparatus according to claim 5, further comprising a blind groove formed in the first-housing lower wall at the fastening portion.

8. The radiation imaging apparatus according to claim 2, further comprising a fastening member configured to fasten the fastening portion,
wherein an end of the fastening member on the side of the at least one side-surface is flush with, or recessed inward from, the outermost surface of the at least one side surface.

9. The radiation imaging apparatus according to claim 3, further comprising a support base configured to support the radiation detector on the rear-surface side,
wherein an end face of the support base on the side of the at least one side-surface is located on an inner surface of the first-housing upper wall, and an upper end of the side wall of the second housing member on the entrance-surface side is spaced toward the rear surface from the support base.

10. The radiation imaging apparatus according to claim 9, further comprising an insulating layer provided on the inner surface of the first-housing upper wall,
wherein the end face of the support base on the side of the at least one side-surface is in contact with the insulating layer.

11. The radiation imaging apparatus according to claim 3, wherein the first-housing upper wall is thinner than at least one of the entrance surface and the first-housing lower wall.

12. The radiation imaging apparatus according to claim 2, wherein the first housing member has a uniform thickness.

13. The radiation imaging apparatus according to claim 2, wherein a thickness of the first housing member is less than 1 mm.

14. The radiation imaging apparatus according to claim 2, wherein the second housing member has higher rigidity than the first housing member.

15. The radiation imaging apparatus according to claim 14, wherein the second housing member is an integral member with uniform thickness that is thicker than the first housing member.

16. The radiation imaging apparatus according to claim 14, wherein the second housing member includes a second-housing rear surface forming the rear surface of the housing and a second-housing side wall thicker than the first housing member.

17. The radiation imaging apparatus according to claim 1, wherein the at least one side surface is a narrow-frame side surface in which a distance from the outmost surface of the housing to an effective imaging region of the radiation detector is less than a distance of other side surfaces of the housing to the effective imaging region of the radiation detector.

18. The radiation imaging apparatus according to claim 1, wherein the apparatus is configured for mammography imaging.
